(19) United States
(12) Patent Application Publication
(10) Pub. No.: US 2023/0041666 A1
(43) Pub. Date: Feb. 9, 2023

(54) AUTONOMOUS WATER QUALITY SENSING APPARATUS, SYSTEM AND METHOD FOR OPERATING THE APPARATUS

(71) Applicant: ESTINET TECHNOLOGIES INCORPORATION, Hsinchu City (TW)

(72) Inventor: CHIH-CHE LIN, Hsinchu City (TW)

(21) Appl. No.: 17/393,374

(22) Filed: Aug. 3, 2021

(51) Int. Cl. G01N 33/18; A01K 63/00; G01N 35/00

(52) U.S. Cl. CPC G01N 33/18; A01K 63/006; G01N 35/00; G01N 2035/1062

(57) ABSTRACT

An autonomous water quality sensing apparatus, a system and a method for operating the apparatus are provided. In the autonomous water quality sensing system, the autonomous water quality sensing apparatus is configured to move on a track. In the method, a driving mechanism is used to drive the autonomous water quality sensing apparatus to operate over an elevated track surrounding one or more pools. The autonomous water quality sensing apparatus includes a sensing device. The sensor is put into the pool at a planned stop by a sensor deploying mechanism of the autonomous water quality sensing apparatus, so as to obtain water quality data of each of the pools according to a routing plan and a length setting.

### FIELD OF THE DISCLOSURE

The present disclosure relates to a technology of sensing water quality, and more particularly to an autonomous water quality sensing system, an apparatus, and a method for operating the apparatus which allows an automatic operation over a route.

### BACKGROUND OF THE DISCLOSURE

Water quality management is essential to the aquaculture industry. Good water quality can help increase fish production, and quality products can be obtained. If there is a problem with the water quality, an aquaculturist may suffer huge losses. Therefore, water quality management has become one of the most important issues for aquaculture.

Parameters relating to the water quality of a fish farm include water temperature, pH value, dissolved oxygen content, turbidity, etc., and various things around the environment may affect the water quality. In the conventional water quality monitoring technologies, various water quality sensors (such as a thermometer, a pH meter, a dissolved oxygen sensor and an optical sensor for measuring turbidity) are relied upon. Furthermore, a remote monitoring technology can be used cooperatively.

FIG. 1 is a schematic diagram depicting a conventional device used to sense the water quality of the fish farm.

In a fish farm 10 shown in the diagram, a buoyant apparatus 101 is utilized to carry a sensing device 103. The buoyant apparatus 101 floats on the water of the fish farm 10, so as to drop the sensing device 103 in the water for sensing the water quality. The buoyant apparatus 101 can float on the fish farm to continuously sense the water quality. However, when the buoyant apparatus 101 needs to be cleaned or used to sense water quality of other fish farms, it will take some time to retract the buoyant apparatus 101 manually. Both the buoyant apparatus 101 and the sensing device 103 are then brought to another place for sensing the water quality of said place in the same manner. Further, if a sensor of the sensing device 103 needs to be cleaned or calibrated, the buoyant apparatus 101 still needs to be retracted manually, so that the sensor can be removed to conduct cleaning and calibration.

### SUMMARY OF THE DISCLOSURE

In response to the above-referenced technical inadequacies, the present disclosure provides an autonomous water quality sensing apparatus, a system and a method for operating the autonomous water quality sensing apparatus.

The autonomous water quality sensing apparatus is automatized to sense water quality of one or more pools, so as to effectively save onsite manpower. The autonomous water quality sensing apparatus of the present disclosure can prevent issues that include human errors and sensing errors caused by difficulties in data calibration due to adoption of multiple sensors.

In one embodiment of the present disclosure, the autonomous water quality sensing system includes the autonomous water quality sensing apparatus. The autonomous water quality sensing apparatus includes a sensing device, and utilizes a sensor deploying mechanism to put the sensing device into one or more pools for collecting water quality data of each pool. The system provides a track, which can be a single-track or a double-track installed above the pools. The autonomous water quality sensing apparatus is driven to travel over the track by a driving mechanism which is configured to plan a route according to an instruction generated by a control circuit. The autonomous water quality sensing apparatus travels to one or more planned stops based on a routing plan for collecting the water quality data.

Further, the autonomous water quality sensing system includes a central control center used to receive the water quality data collected by the autonomous water quality sensing apparatus at different places.

The system further includes a station which is disposed at a fixed place over the track. When the autonomous water quality sensing apparatus moves to the station, maintenance of the autonomous water quality sensing apparatus can be performed in the station, so as to ensure normal operation thereof.

The autonomous water quality sensing apparatus includes a control circuit that controls a sensor deploying mechanism to put the sensing device in the pools for collecting water quality data and to retract the sensing device. The apparatus also includes a communication circuit for transmitting the water quality data to a communication receiving device when the water quality data is obtained. The communication receiving device can then transmit the water quality data to a central control center.

Preferably, in the autonomous water quality sensing system, the sensor deploying mechanism of the autonomous water quality sensing apparatus puts the sensing device into the pools according to one or more length settings, so as to correctly collect the water quality data in one or more depths of the pools. The routing plan includes the one or more planned stops over the track. Each of the planned stops is configured to include the one or more length settings for deploying the sensing device.

When the autonomous water quality sensing apparatus finishes collection of the water quality data of a specific pool, the control circuit controls the driving mechanism to drive the autonomous water quality sensing apparatus to move to a next planned stop for collecting the water quality data at said stop according to the routing plan.

In one embodiment of the method for operating the autonomous water quality sensing apparatus, in the beginning, the autonomous water quality sensing apparatus is activated to be driven by the driving mechanism, so as to operate on the track according to instructions generated by the control circuit.

According to the mentioned routing plan, the autonomous water quality sensing apparatus is driven to the planned stops for acquiring the water quality data of the pools.

Further, when the water quality data is obtained, the water quality data can be updated and transmitted to the communication receiving device. The communication receiving device transmits the water quality data to the central control center.

Still further, after the water quality data is collected at each of the planned stops, the sensor deploying mechanism is configured to retract the sensing device according to instructions generated by the control circuit. The autonomous water quality sensing apparatus is then driven by the driving mechanism to move to the next planned stop for collecting the water quality data.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic diagram depicting a sensing device in a conventional fish farm;

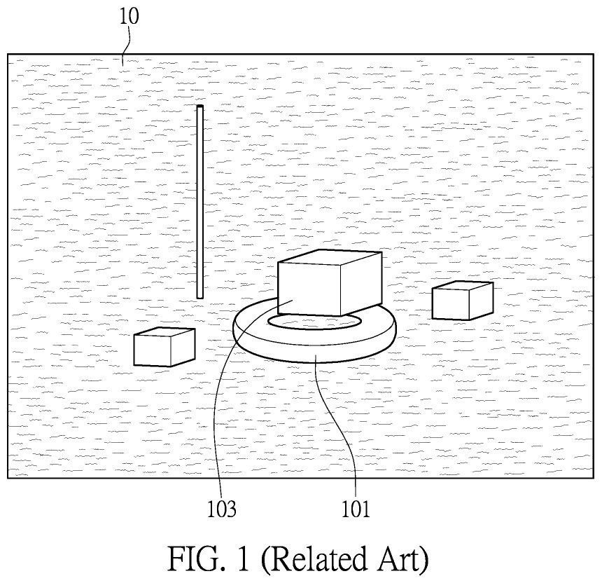

FIG. 2 is a schematic diagram depicting a structure of an autonomous water quality sensing apparatus according to one embodiment of the present disclosure;

FIG. 3 is a block diagram depicting circuit units of the autonomous water quality sensing apparatus according to one embodiment of the present disclosure;

FIG. 4 is a schematic diagram depicting a framework of an autonomous water quality sensing system that operates the autonomous water quality sensing apparatus according to one embodiment of the present disclosure;

FIG. 5 is a flow chart describing a method for operating the autonomous water quality sensing apparatus according to one embodiment of the present disclosure;

FIG. 6 is a schematic diagram depicting operation of the autonomous water quality sensing apparatus according to a first embodiment of the present disclosure;

FIG. 7 is a schematic diagram depicting operation of the autonomous water quality sensing apparatus according to a second embodiment of the present disclosure;

FIG. 8 is a schematic diagram depicting operation of the autonomous water quality sensing apparatus according to a third embodiment of the present disclosure;

FIG. 9 is a schematic diagram depicting operation of the autonomous water quality sensing apparatus according to a fourth embodiment of the present disclosure;

FIG. 10 is a schematic diagram depicting operation of the autonomous water quality sensing apparatus according to a fifth embodiment of the present disclosure; and

FIG. 11 is a schematic diagram depicting operation of the autonomous water quality sensing apparatus according to a sixth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The present disclosure is related to an autonomous water quality sensing apparatus, a system and an operating method. The autonomous water quality sensing apparatus is a device that is capable of moving autonomously for water quality sensing. In addition to a main body of the apparatus, various sensors for obtaining different kinds of water quality data are disposed inside the apparatus. A sensing device can be used to contain the one or more sensors. The sensing device is controlled by a control circuit for obtaining the water quality data. The water quality data can be stored in the apparatus or be transmitted to an external device via a communication circuit.

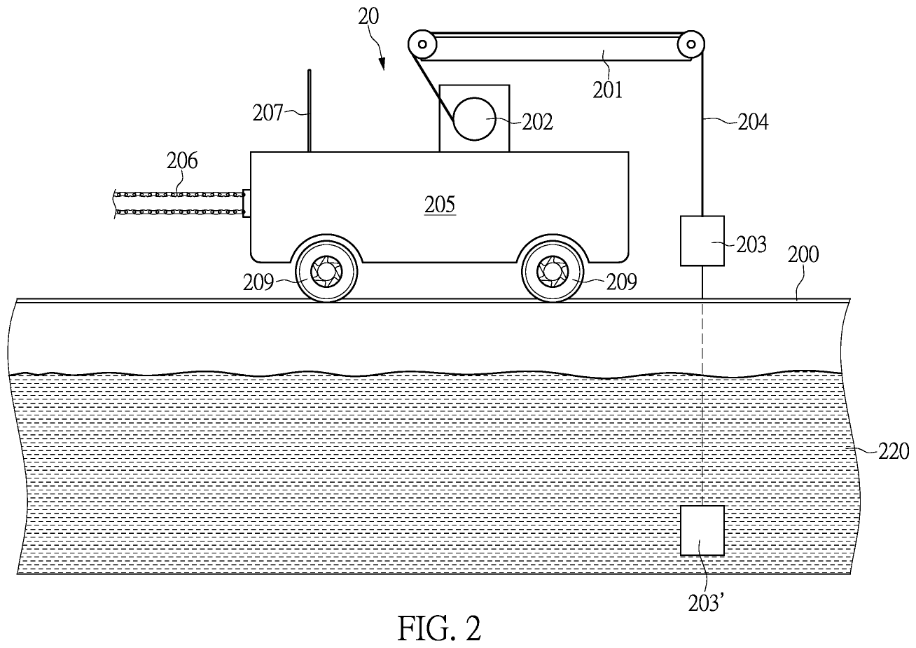

FIG. 2 is a schematic diagram depicting a structure of the autonomous water quality sensing apparatus according to one embodiment of the present disclosure.

The diagram shows the main structural elements of an autonomous water quality sensing apparatus 20. A main body 205 of the autonomous water quality sensing apparatus 20 includes, but is not limited to, a control circuit, a battery, a data-processing circuit, etc. The autonomous water quality sensing apparatus 20 includes a communication circuit that supports a specific communication protocol. The communication circuit transmits the data to an external device via an antenna 207. Alternatively, the autonomous water quality sensing apparatus 20 can transmit signals via a drive chain 206.

According to one embodiment of the present disclosure, the autonomous water quality sensing apparatus 20 can be driven by its autonomous driving mechanism 209 (e.g., a wheel or a tube) to move on a ground or to operate over a track 200. In an exemplary example, an internal driver of the autonomous water quality sensing apparatus 20 drives the driving mechanism 209 to operate over the track 200; alternatively, the driving mechanism 209 can be the drive chain 206 of the autonomous water quality sensing apparatus 20, so as to drive the autonomous water quality sensing apparatus 20 to operate over the track 200. It should be noted that the driving mechanism 209 is not limited to the above-mentioned types. A routing plan allows the autonomous water quality sensing apparatus 20 to autonomously stop at one or more planned stops for sensing water quality. In the diagram, a sensor deploying mechanism 201 controls a rope 204 to put a sensing device 203 into water 220 according to a length setting. A sensing device 203′ put in the water 220 is configured to sense the water quality according to a control signal in the main body 205.

In one embodiment of the present disclosure, the sensor deploying mechanism 201 of the autonomous water quality sensing apparatus 20 includes a winder 202 and a rope 204 shown in the diagram. The rope 204 is used to fasten the sensing device 203. The sensor deploying mechanism 201 can be a robot arm or a specific lever mechanism that puts the sensing device 203 into the water 220 via the winder 202 and the rope 204 that is driven by the winder 202. According to the length setting, the sensing device 203 is put into the pools at the planned stops. The sensing device 203 can be a sensor used for sensing specified water quality data, or an assembly that integrates one or more sensors. The sensors in the sensing device 203 can be a pH sensor, a thermal sensor, a conductivity sensor and a dissolved oxygen content sensor.

A data-processing circuit inside the main body 205 of the autonomous water quality sensing apparatus 20 is, for example, a central processor used to operate an operating system (or system software that is able to drive the circuits) for processing the acquired water quality data. The water quality data can be stored in a memory of the autonomous water quality sensing apparatus 20, or transmitted to an external device via the communication circuit. The autonomous water quality sensing apparatus 20 includes a control circuit. The control circuit is used to generate control instructions for operating circuits and mechanism of the autonomous water quality sensing apparatus 20. For example, the above-mentioned driving mechanism 209 is controlled by the control circuit to drive the autonomous water quality sensing apparatus 20 to move to one of the planned stops, so as to put the sensing device 203 for sensing water quality in a specific depth by the winder 202 or the rope 204. The autonomous water quality sensing apparatus 20 performs tasks such as collecting data, storing the data, transmitting the data to an external device, and retracting the sensing device 203.

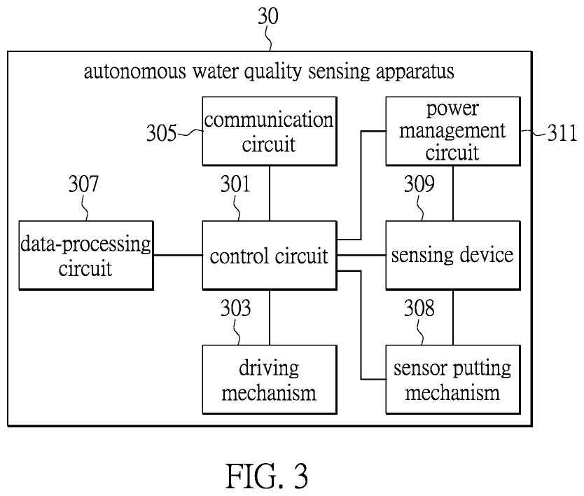

Reference is made to FIG. 3, which is a schematic diagram depicting functional modules that include circuits and the mechanism of the autonomous water quality sensing apparatus according to one embodiment of the present disclosure.

According to the embodiment, an autonomous water quality sensing apparatus 30 includes a control circuit 301 that is electrically connected with the circuit components of the autonomous water quality sensing apparatus 30, so as to control the operations of the components. The autonomous water quality sensing apparatus 30 essentially includes a sensing device 309 which is electrically connected with the control circuit 301. The sensing device 309 is mounted on a sensor deploying mechanism 308, which is configured to put the sensing device 309 into one or more pools for collecting the water quality data according to commands issued by the control circuit 301.

The sensing device 309 can be a specific sensor, or preferably a device integrating multiple types of sensors. The autonomous water quality sensing apparatus 30 includes a driving mechanism 303 which can be, but not limited to, a driving wheel or a caterpillar band. The driving mechanism 303 is electrically connected with the control circuit 301. The driving mechanism 303 drives the autonomous water quality sensing apparatus 30 to operate over the track in response to instructions generated by the control circuit 301. The autonomous water quality sensing apparatus 30 is driven to move to one of the planned stops for collecting the water quality data of the pools according to a pre-determined routing plan specific to a site.

It should be noted that the routing plan includes control parameters set by an administrator that are specific to the site for water quality sensing. For example, the control parameters of the routing plan can include the planned stops over the track, depths for putting the sensing device 30, the time for sensing water quality at each of the planned stops, the data indicative of water quality, and even settings for regular cleaning of the autonomous water quality sensing apparatus 30.

In one of the embodiments, the autonomous water quality sensing system includes a station used for maintenance. The routing plan can also include routes and times that the autonomous water quality sensing apparatus 30 is controlled to enter the station for maintenance (such as cleaning and data transmission).

The autonomous water quality sensing apparatus 30 further includes a communication circuit 305 that is electrically connected with the control circuit 301. The communication circuit 305 is in compliance with a specific communication protocol. When the water quality data is obtained, the communication circuit 305 is used to transmit in real time the water quality data to a communication receiving device with the specific communication protocol. The communication receiving device then transmits the data back to the central control center.

The autonomous water quality sensing apparatus 30 puts the sensing device 309 into the pools by the sensor deploying mechanism 308 according to instructions generated by the control circuit 301. The instructions can include one or more length settings at each of the planned stops. The sensing device 309 is put into the pools for collecting the water quality data at one or more depths based on the instructions.

The autonomous water quality sensing apparatus 30 includes a data-processing circuit 307. The data-processing circuit 307 processes the data after the autonomous water quality sensing apparatus 30 collects the water quality data by the sensing device 309. By the data-processing circuit 307, the data is preliminarily processed and then stored to a memory of the autonomous water quality sensing apparatus 30, or can be sent to an external device via the communication circuit 305 in real time.

The autonomous water quality sensing apparatus 30 includes a power management circuit 311 that is used for managing power supplied to the circuit components and mechanical components of the apparatus 30 via electrical connections. In one of the embodiments, the power management circuit 311 is used to manage operations of a battery set of the apparatus 30. In one further embodiment, the autonomous water quality sensing apparatus 30 can also be powered via the energized track. Moreover, when the power management circuit 311 determines that the electrical power supplied to the autonomous water quality sensing apparatus 30 has declined to a specific threshold, the control circuit 301 can generate instructions for controlling the driving mechanism 303 to drive the autonomous water quality sensing apparatus 30 to move to the station for charging.

According to certain embodiments of the present disclosure, the autonomous water quality sensing apparatus 30 can operate over the track. The track can be a monorail track or a dual rail track that is installed above the pools (or a target to be sensed). In addition to using wheels for the autonomous water quality sensing apparatus 30 to move over the track, a rope can also be used to pull the autonomous water quality sensing apparatus 30 to move.

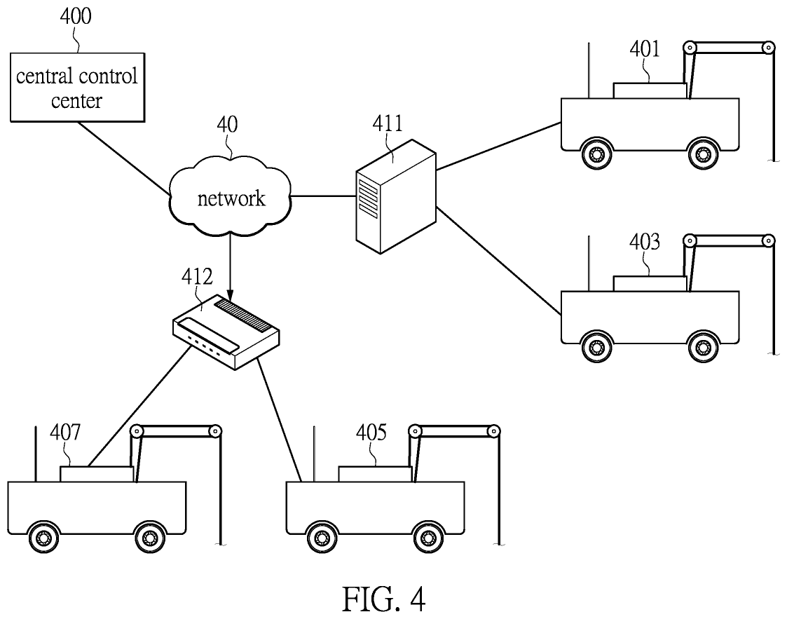

FIG. 4 is a schematic diagram depicting a framework of a water quality sensing system operating the autonomous water quality sensing apparatus according to one embodiment of the present disclosure.

The water quality sensing system shown in the diagram includes a central control center 400, which is able to connect with multiple autonomous water quality sensing apparatuses 401, 403, 405 and 407 at different places via a network 40. The central control center 400 receives water quality data collected by the autonomous water quality sensing apparatuses 401, 403, 405 and 407 at the different places.

It should be noted that more than one autonomous water quality sensing apparatus can operate at one site. For example, the autonomous water quality sensing apparatuses 401 and 403 shown in the diagram can be used at one site simultaneously. A gateway 411 linked to the network 40 is provided for transmitting data to the central control center 400. The autonomous water quality sensing apparatuses 405 and 407 are linked to the network 40 via a gateway 412. The gateway 411 or 412 operates as a communication receiving device that is configured to receive the water quality data collected by the autonomous water quality sensing apparatuses 401, 403, 405 and 407 in real time. The water quality data can also be transmitted to the central control center 400 after being processed in the autonomous water quality sensing apparatuses 401, 403, 405 and 407. The central control center 400 can remotely monitor the water quality of different sites. In one of the embodiments, the central control center 400 can remotely set up and operate the autonomous water quality sensing apparatuses 401, 403, 405, and 407.

The autonomous water quality sensing system provides at least one autonomous water quality sensing apparatus at each site. The sensing device of the apparatus is used to sense the water quality of the pools. In the system, the track is provided for the autonomous water quality sensing apparatus to operate. More particularly, the apparatus is to operate according to a routing plane for each site. Accordingly, the autonomous water quality sensing apparatus can regularly move to the planned stops for acquiring the water quality data of the pools.

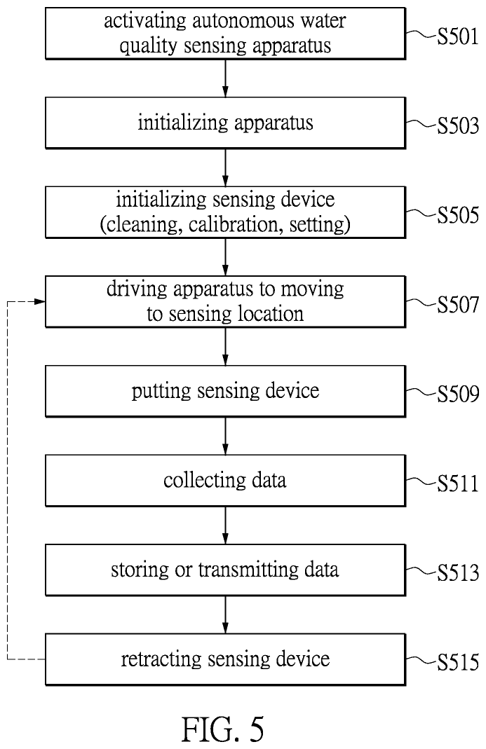

According to one embodiment of the present disclosure, operations of the autonomous water quality sensing apparatus (as shown in a flow chart of FIG. 5) are based on a routing plan and a length setting at each of the planned stops.

In the beginning, as shown in step S501, the autonomous water quality sensing apparatus is placed on a track and then activated. In step S503, after activation, the autonomous water quality sensing apparatus is initialized. An operating system running in the apparatus confirms that the electrical power, the circuit components and the mechanism of the apparatus can operate normally. Further, in step S505, the sensing device of the autonomous water quality sensing apparatus is also initialized for the purpose of cleaning, calibration and initial setting.

Next, in step S507, the operating system running in the autonomous water quality sensing apparatus generates instructions via the control circuit. The control circuit controls the driving mechanism to drive the autonomous water quality sensing apparatus to move to a sensing location. In step S509, the sensor deploying mechanism is configured to put the sensing device into one of the pools in a specific depth according to the length setting. In step S511, the sensing device starts to collect water quality data of each of the pools. In step S513, the water quality data is stored or transmitted to an external device (such as a station or a central control center). In step S515, the control circuit controls the sensor deploying mechanism to retract the sensing device.

If the control circuit determines that there is a next planned stop, the control circuit controls the autonomous water quality sensing apparatus to move to the next planned stop for performing the steps S507 to S515.

In the following steps, in the station or the central control center, the water quality data transmitted from the autonomous water quality sensing apparatus is analyzed. A report is produced and transmitted to a device of the administrator of the pools. For example, an aquaculturist can use the autonomous water quality sensing system of the present disclosure to acquire the water quality data, the report or even an alert through a software program provided thereby.

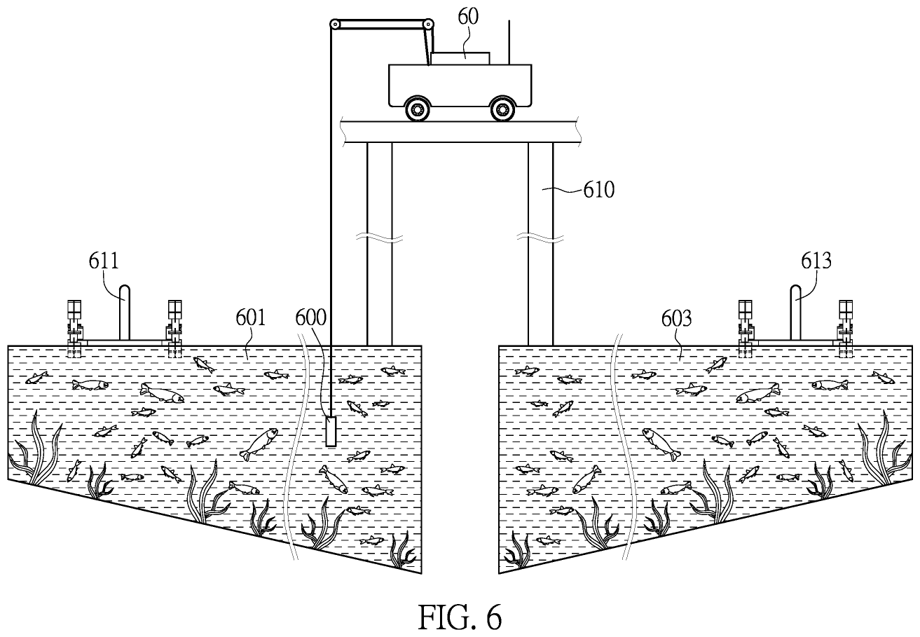

The autonomous water quality sensing apparatus can be applied to sites as described below. Reference is made to FIG. 6, which is a schematic diagram depicting operation of the autonomous water quality sensing apparatus at a specific site according to one embodiment of the present disclosure.

Two pools 601 and 603 are shown in the diagram, and two splashing devices 611 and 613 can be disposed in the pools 601 and 603 (e.g., fish farm), respectively. The autonomous water quality sensing system can be used to acquire the water quality data at the site.

An autonomous water quality sensing apparatus 60 operates over an elevated track 610. The elevated track 610 is installed between the two pools 601 and 603, so as to allow the autonomous water quality sensing apparatus 60 to acquire the water quality data of the two pools 601 and 603. In one embodiment of the present disclosure, the system can provide different schemes to meet different requirements of the pools 601 and 603. For example, the system can provide one or more length settings specific to each of the pools. Accordingly, the autonomous water quality sensing apparatus 60 controls the sensor deploying mechanism to put the sensing device 600 into the pools 601 and 603 in a specific length, so as to collect the water quality data in one or more depths.

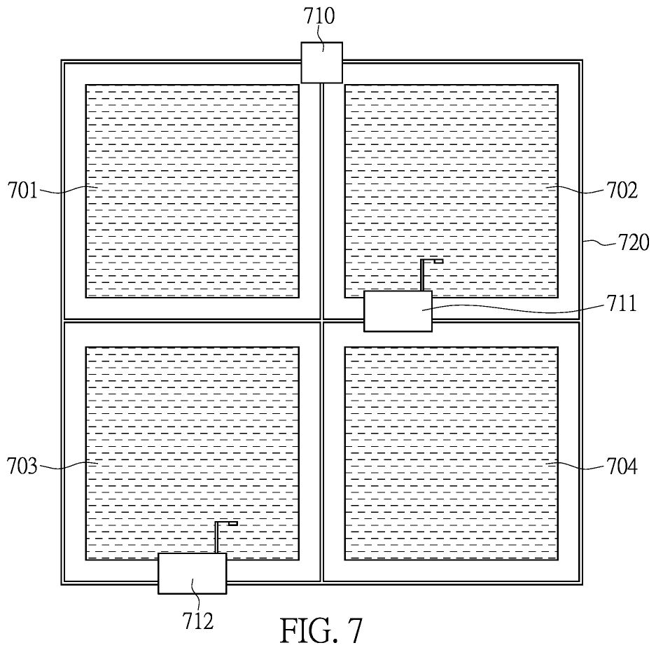

FIG. 7 is a schematic diagram depicting operation of the autonomous water quality sensing apparatus according to one embodiment of the present disclosure. Multiple pools 701, 702, 703 and 704 are provided. An aquaculture site can include one or more of these pools. A track 720 is installed to surround these pools 701, 702, 703 and 704 in a circular manner, and the track 720 can be an elevated design in one of the embodiments. A routing plan arranged to include multiple planned stops is applied to the track 720.

More than one autonomous water quality sensing apparatus can be used on the track 720. In the present exemplary example, a first autonomous water quality sensing apparatus 711 and a second autonomous water quality sensing apparatus 712 are provided. Each of the autonomous water quality sensing apparatuses 711 and 712 operates on the track 720 according to their respective routing plan. A station 710 is disposed at a fixed location of the track 720. The first autonomous water quality sensing apparatus 711 and the second autonomous water quality sensing apparatus 712 can each move to the station 710 for maintenance. For example, the apparatuses and the sensors can be cleaned, maintained and charged in the station 710. Further, the water quality data can also be transmitted in the station 710.

In one embodiment of the present disclosure, the station 710 includes a data host that is used to retrieve the water quality data obtained by the autonomous water quality sensing apparatus. The communication circuit of the apparatus can be used to transmit the water quality data to the station 710 or the central control center in real time.

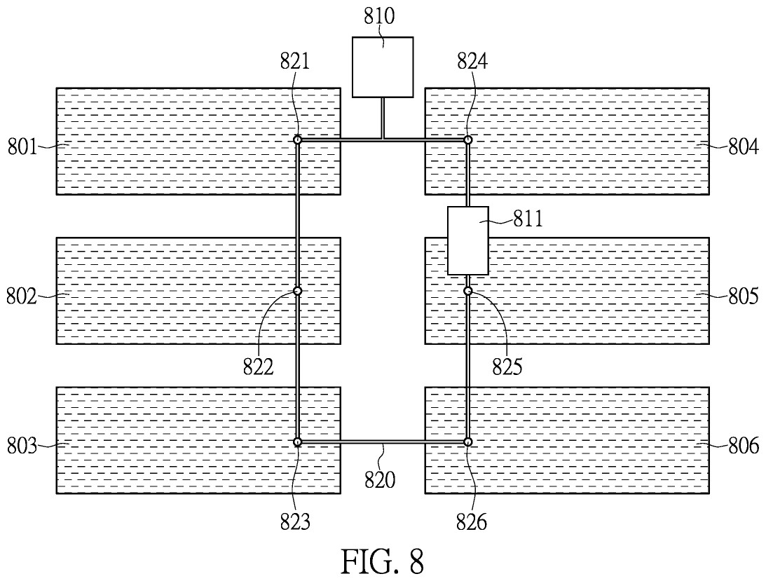

FIG. 8 is a schematic diagram depicting operation of the autonomous water quality sensing apparatus according to one embodiment of the present disclosure. A site shown in the diagram includes multiple pools 801, 802, 803, 804, 805 and 806 that are arranged in an array. A track 820 surrounding these pools 801, 802, 803, 804, 805 and 806 is provided. An autonomous water quality sensing apparatus 811 moves on the track 820 in a circular manner. A station 810 is disposed on the track 820.

Multiple planned stops 821, 822, 823, 824, 825 and 826 that respectively correspond to the multiple pools 801, 802, 803, 804, 805 and 806 are arranged on the track 820. For each of the planned stops, one or more length settings for placing the sensing device are applied. Both the length settings and the routing plan that covers the planned stops 821, 822, 823, 824, 825 and 826 are stored in a memory of the autonomous water quality sensing apparatus 811. The control circuit controls the autonomous water quality sensing apparatus 811 to perform water quality sensing according to the routing plan and the length setting of each of the planned stops. For example, the sensor deploying mechanism of the autonomous water quality sensing apparatus 811 puts the sensing device into the pools 801, 802, 803, 804, 805 and 806 for sensing water quality in one or more depths according to the length settings.

When the autonomous water quality sensing apparatus 811 completes the data collection, the control circuit controls the driving mechanism to drive the autonomous water quality sensing apparatus 811 to move to a next planned stop for sensing water quality according to the routing plan.

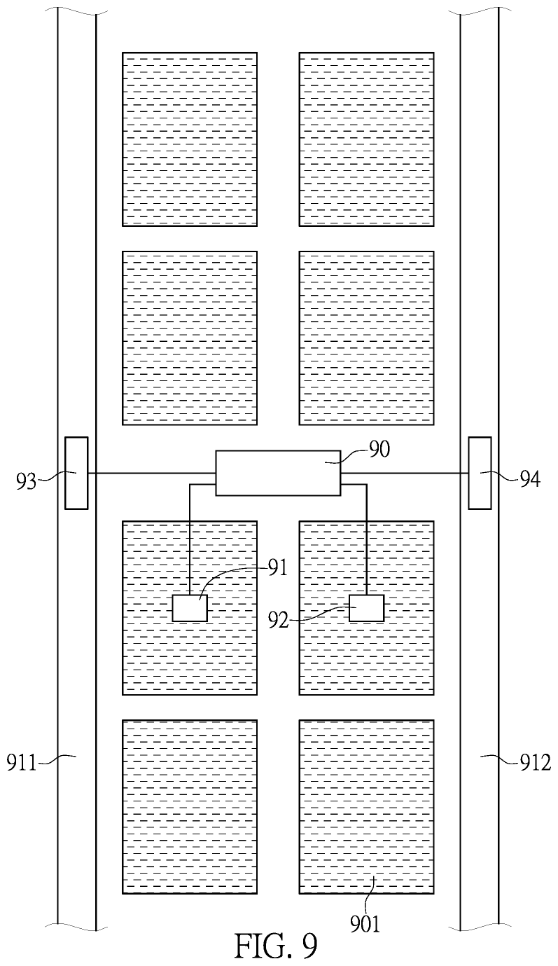

Reference is made to FIG. 9, which shows another site to which the autonomous water quality sensing apparatus is applicable. Multiple pools 901 are provided at the site. In order to perform water quality sensing operations efficiently and automatically, two parallel tracks 911 and 912 are provided above the pools 901. An autonomous water quality sensing apparatus 90 uses one or more cantilevers to hang a sensing device with a rope. In the present example, two of the cantilevers hang two sensing devices 91 and 92 for sensing water quality of two of the pools 901 at the same time. Two driving mechanism 93 and 94 are respectively installed on the tracks 911 and 912 for driving the autonomous water quality sensing apparatus 90 to move around the pools 901 in a certain linear route.

In accordance with the operations described in FIG. 5, the autonomous water quality sensing apparatus 90 operates based on a planned route. The system sets up the planned stops for the autonomous water quality sensing apparatus 90, as well as time, sensing items and lengths for putting the sensing devices 91 and 92 at each of the pools 901. These settings are stored in the memory of the autonomous water quality sensing apparatus 90. An operating system running in the autonomous water quality sensing apparatus 90 and the control circuit control the driving mechanism 93 and 94 to drive the apparatus 90 to automatically move to multiple sensing locations, and proceed with the sensing tasks that include putting the sensing device 91 and 92 in the pools 901.

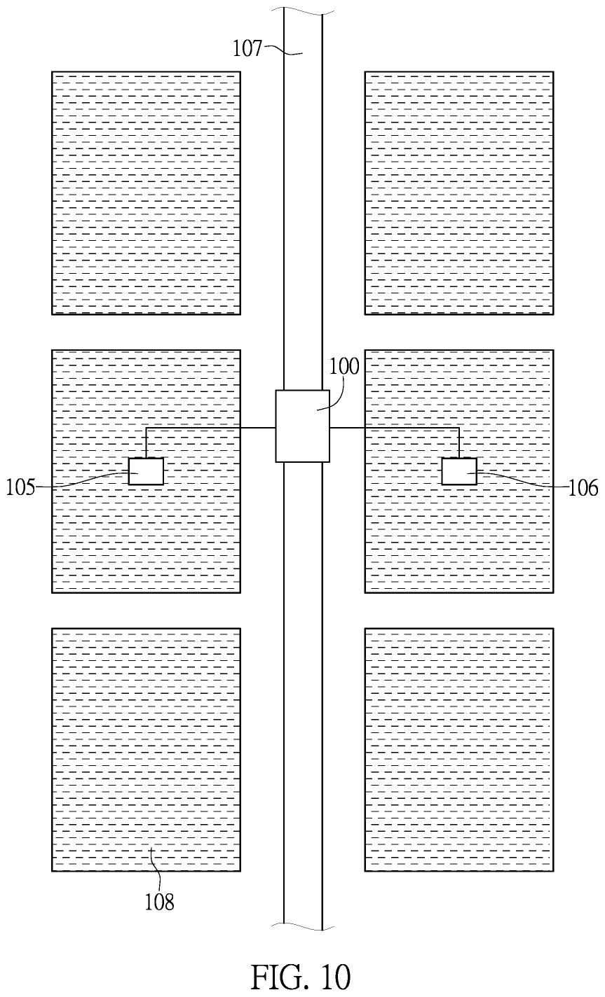

FIG. 10 is a schematic diagram depicting operation of the autonomous water quality sensing apparatus in one further embodiment of the present disclosure. A monorail track 107 installed above multiple pools 108 is provided, and an autonomous water quality sensing apparatus 100 operates over the track 107. The autonomous water quality sensing apparatus 100 uses one or more cantilevers to hang and put a sensing device into the pools 108 for sensing the water quality. The autonomous water quality sensing apparatus 100 of the present example includes two cantilevers which hang from two sensing devices 105 and 106 respectively. The driving mechanism of the autonomous water quality sensing apparatus 100 drives the autonomous water quality sensing apparatus 100 to move on the track 107 in a linear direction, and puts the sensing devices 105 and 106 into the pools 108 for sensing the water quality at the planned stops.

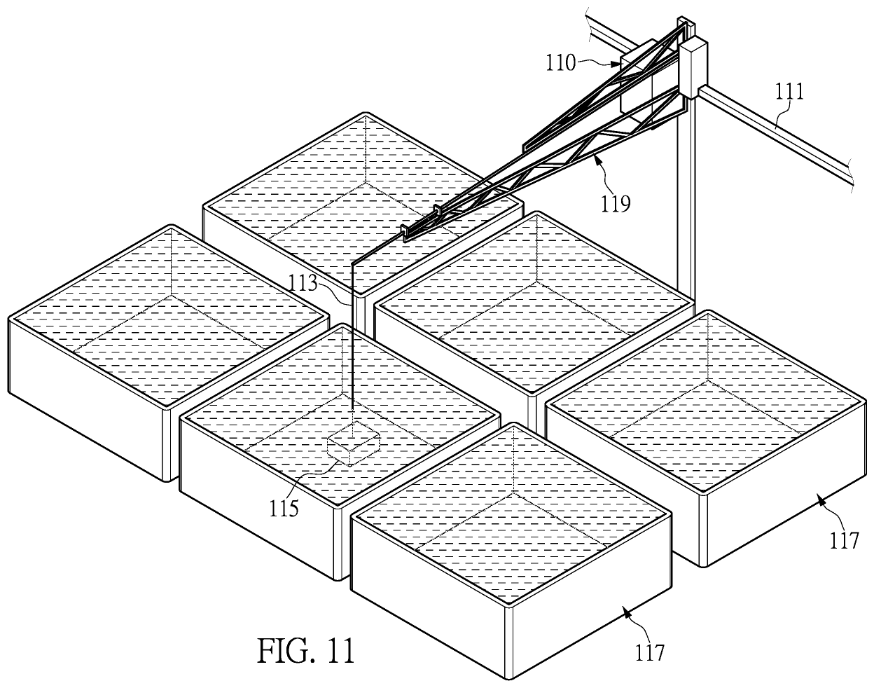

Reference is made to FIG. 11, which is a schematic diagram depicting operation of the autonomous water quality sensing apparatus according to one embodiment of the present disclosure. A sensing system including a single-arm autonomous water quality sensing apparatus 110 is shown. The autonomous water quality sensing apparatus 110 moves over the track 111 by a driving mechanism. It should be noted that, in addition to the driving mechanism driving the autonomous water quality sensing apparatus 110 to move, the autonomous water quality sensing apparatus 110 further relies on a mechanical design to move over the track 111 stably. A control circuit controls a cantilever 119 to be stretched and rotated, and controls a length of a rope 113, so as to put a hanging sensing device 115 into each of pools 117 for sensing water quality. According to one embodiment of the present disclosure, the autonomous water quality sensing apparatus 110 utilizes the cantilever 119 to hang the sensing device 115 via the rope 113. The autonomous water quality sensing apparatus 110 is driven by the driving mechanism to stop at each of the planned stops according to the routing plan and sensing settings. A rotating angle and a stretching length of the cantilever 119 are controlled for operating the hanging sensing device 115 into the pools 117. The autonomous water quality sensing apparatus 110 can therefore be controlled for effectively sensing water quality of each of the pools 117 at the site.

In conclusion, according to the above embodiments of the autonomous water quality sensing system, the apparatus and the operating method, the autonomous water quality sensing apparatus can be commonly used for multiple pools. As the autonomous water quality sensing apparatus can be operated in each of the sites automatically, the sensing device can arrive at a specific location at a specific time to perform a sensing operation automatically. In this way, the automatic control operation effectively prevents human errors, and can also prevent pollution and sensing errors caused by the sensor which is put into the water (or a specific environment) for a long time. Another advantage is that, since one autonomous water quality sensing apparatus is shared by the multiple pools, errors among the sensors that are used in the multiple pools can also be prevented.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An autonomous water quality sensing system, comprising:
an autonomous water quality sensing apparatus including a sensing device, wherein the autonomous water quality sensing apparatus utilizes a sensor deploying mechanism to drop the sensing device into one or more pools for collecting water quality data of the one or more pools; and
a track, wherein the autonomous water quality sensing apparatus is driven by a driving mechanism to travel over the track, and to stop at one or more planned stops for acquiring the water quality data of each of the pools according to a routing plan.

2. The autonomous water quality sensing system according to claim 1, further comprising a central control center which is used to receive the water quality data collected by multiple ones of the autonomous water quality sensing apparatus disposed at different places.

3. The autonomous water quality sensing system according to claim 1, further comprising a station disposed at a fixed location over the track, wherein the autonomous water quality sensing apparatus moves to the station for maintenance.

4. The autonomous water quality sensing system according to claim 1, wherein the autonomous water quality sensing apparatus includes a control circuit used to control the sensor deploying mechanism to put the sensing device into each of the pools, and to retract the sensing device after collection of the water quality data is completed.

5. The autonomous water quality sensing system according to claim 4, wherein the autonomous water quality sensing apparatus includes a communication circuit electrically connected with the control circuit; wherein the communication circuit is used to transmit the water quality data to a communication receiving device in real time when the water quality data is obtained, and the water quality data is forwarded to a central control center by the communication receiving device.

6. The autonomous water quality sensing system according to claim 1, wherein the sensor deploying mechanism of the autonomous water quality sensing apparatus puts the sensing device into the pools according to one or more length settings, so as to collect the water quality data at one or more depths of each of the pools.

7. The autonomous water quality sensing system according to claim 6, wherein the routing plan includes the one or more planned stops over the track, and each of the planned stops is configured to include the one or more length settings for deploying the sensing device.

8. The autonomous water quality sensing system according to claim 7, wherein, when collection of the water quality data is completed by the autonomous water quality sensing apparatus, the control circuit controls the driving mechanism to drive the autonomous water quality sensing apparatus to move to a next one of the planned stops for collecting the water quality data according to the routing plan.

9. The autonomous water quality sensing system according to claim 8, wherein the track is an elevated monorail track or an elevated dual rail track installed above the one or more pools that are located at a same location.

10. The autonomous water quality sensing system according to claim 9, wherein the track is a circular track or a linear track surroundingly arranged above the one or more pools.

11. The autonomous water quality sensing system according to claim 10, wherein one or multiple ones of the sensing device are hung on one or more cantilevers of the autonomous water quality sensing apparatus, and put the one or multiple ones of the sensing device into the one or more pools for sensing the water quality.

12. An autonomous water quality sensing apparatus, comprising:
a control circuit electrically connected to circuit units of the autonomous water quality sensing apparatus, so as to control operations of the circuit units;
a sensing device electrically connected to the control circuit and coupled to a sensor deploying mechanism, wherein, according to instructions of the control circuit, the sensor deploying mechanism puts the sensing device into one or more pools for collecting water quality data of the one or more pools; and
a driving mechanism electrically connected to the control circuit, wherein the driving mechanism is used to drive the autonomous water quality sensing apparatus to operate on a track according to the instructions of the control circuit, and drive the autonomous water quality sensing apparatus to move to one or more planned stops for acquiring the water quality data of each of the pools according to a routing plan.

13. The autonomous water quality sensing apparatus according to claim 12, wherein the driving mechanism is driven by an internal driver of the autonomous water quality sensing apparatus.

14. The autonomous water quality sensing apparatus according to claim 12, wherein the driving mechanism is a drive chain which is used to drive the autonomous water quality sensing apparatus to move.

15. The autonomous water quality sensing apparatus according to claim 12, further comprising a communication circuit electrically connected with the control circuit, wherein the communication circuit is used to transmit the water quality data to a communication receiving device in real time when the water quality data is obtained, and the water quality data is forwarded to a central control center by the communication receiving device.

16. The autonomous water quality sensing apparatus according to claim 12, wherein the sensor deploying mechanism of the autonomous water quality sensing apparatus puts the sensing device into the pools according to one or more length settings, so as to collect the water quality data at one or more depths of each of the pools.

17. The autonomous water quality sensing apparatus according to claim 16, wherein the sensor deploying mechanism includes a winder and a rope, and the rope fixes the sensing device, so as to put the sensing device into the pools.

18. A method for operating an autonomous water quality sensing apparatus, comprising:
activating the autonomous water quality sensing apparatus, wherein a driving mechanism is used to drive the autonomous water quality sensing apparatus to operate over a track according to instructions generated by a control circuit; wherein the autonomous water quality sensing apparatus includes a sensing device, and utilizes a sensor deploying mechanism to drop the sensing device in one or more pools for collecting water quality data of the one or more pools; wherein the autonomous water quality sensing apparatus is driven to stop at one or more planned stops for acquiring the water quality data of each of the pools according to a routing plan.

19. The method according to claim 18, wherein the autonomous water quality sensing apparatus includes a communication circuit electrically connected with the control circuit; wherein the communication circuit is used to transmit the water quality data to a communication receiving device in real time when the water quality data is obtained, and the water quality data is forwarded to a central control center by the communication receiving device.

20. The method according to claim 19, wherein, when collection of the water quality data is completed by the autonomous water quality sensing apparatus, according to instructions generated by the control circuit, the sensor deploying mechanism is configured to retract the sensing device, and the driving mechanism is configured to drive the autonomous water quality sensing apparatus to move to a next one of the planned stops for collecting the water quality data.
